(19) United States
(12) Patent Application Publication
(10) Pub. No.: US 2009/0254053 A1
(43) Pub. Date: Oct. 8, 2009

(54) IRRIGATION DRESSING AND METHOD OF APPLYING SUCH AN IRRIGATING DRESSING

(75) Inventors: Anna Svensby, Vastra Frolunda (SE); Ulf Johannison, Landvetter (SE); Tomas Fabo, Molnlycke (SE); Bengt Soderstrom, Molndal (SE); Angelica Andresen, Torslanda (SE)

Correspondence Address: YOUNG & THOMPSON, 209 Madison Street, Suite 500, ALEXANDRIA, VA, 22314, US

(73) Assignee: MOLNLYCKE HEALTH CARE AB, GOTEBORG (SE)

(21) Appl. No.: 12/305,590

(22) PCT Filed: Jul. 2, 2007
(86) PCT No.: PCT/SE07/50484

§ 371 Date: Feb. 20, 2009

(30) Foreign Application Priority Data
Jul. 11, 2006 (SE) 0601536-6

(51) Int. Cl. A61M 27/00; A61F 13/02

(52) U.S. Cl. 604/290; 604/305

(57) ABSTRACT

The present invention relates to an irrigation dressing, comprising a first body (5) of a soft, liquid-permeable material, at least one connection (8) for the supply of fluid, at least one connection (9) for the drainage of fluid, an airtight and liquid-tight covering layer (10) of flexible material, which covers said material body and extends outside the sides thereof, and an element (2, 3) for affixing the area of the covering layer that extends outside the contour of the first material body to the skin, the first material body facing the wound bed (W) when the dressing is applied. According to the invention the element (2, 3) for affixing said area to the skin includes a layer (3) of a soft, skin-friendly adhesive, which affords sealing against micro-leakage. The invention also relates to a method of applying such a dressing.

### TECHNICAL FIELD

The present invention relates to an irrigation dressing, comprising a first body of a soft, liquid-permeable material, at least one connection for the supply of fluid, at least one connection for the drainage of fluid, an airtight and liquid-tight covering layer of flexible material, which covers said material body and extends outside the sides thereof, and means for affixing the area of the covering layer that extends outside the contour of the first material body to the skin, the first material body facing the wound bed when the dressing is applied; and to a method of applying such a dressing to a wound.

### BACKGROUND ART

WO 2006/046060 discloses an irrigation dressing, in which the irrigation is performed by a supply from a drip bottle with simultaneous drainage by generating a negative pressure in the drainage connection, using a vacuum pump. One disadvantage of such a dressing is that, in order for the dressing to function, control elements must be provided for continuously regulating the pumping action. One reason for the continuous regulation of the pump is that the known irrigation dressing cannot reliably prevent the ingress of air.

S-B-440 314 demonstrates an irrigation dressing, which in one embodiment specifies how a patient himself can regulate the irrigation process by controlling the drip bottle and manual vacuum pump. The dressing disclosed by said Swedish patent comprises the characteristic features specified in the introductory part.

The object of the present invention is primarily to improve the irrigation dressing disclosed by SE-B-440 314 by providing an irrigation dressing which is easy to handle and permits automatic cleaning of wounds during its period of application without intervention by the patient or staff, which has an improved flow of fluid in the dressing, and which permits an integral dressing of disposable type incorporating all constituent components.

### DISCLOSURE OF INVENTION

These objects are achieved by means of an irrigation dressing comprising a first body of a soft, liquid-permeable material, at least one connection for the supply of fluid, at least one connection for the drainage of fluid, an airtight and liquid-tight covering layer of flexible material, which covers said material body and extends outside the sides thereof, and means for affixing the area of the covering layer that extends outside the contour of the first material body to the skin, the first material body facing the wound bed when the dressing is applied, characterized in that the means for affixing said area to the skin includes a layer of a soft, skin-friendly adhesive, which affords sealing against micro-leakage. The fact that the dressing thus affords sealing against micro-leakage means that there is no risk of air infiltrating under the covering layer via skin fissures or other irregularities in the skin and cancelling a negative pressure that has formed, thereby spoiling the working of the dressing. The constituent components of the dressing can easily be designed so that a negative pressure, once generated, is sufficient to maintain an irrigation process until such time as the dressing needs to be changed because the irrigation fluid is used up. Because no regulation of a vacuum pump is required after the initial generation of a negative pressure, the dressing functions automatically throughout its period of application and does not need to be monitored. This absence of any need for regulation after generating a negative pressure in the vacuum pump means that simple types of vacuum pumps can be used, such as a manual bellows pump.

In a preferred embodiment said adhesive has a softness of more than 10 mm and a weight per unit area of at least 50 g/m². The adhesive may be leakproof according to the MHC leakage test with a groove depth of 75 micrometers.

A membrane of liquid-tight material is preferably arranged between the covering layer and the first material body, one or more connections for the supply of fluid opening out between the membrane and the first material body, one or more connections for the drainage of fluid opening out between the covering layer and the membrane, and the space between the covering layer and the membrane being connected to the space between the membrane and the first material body via at least one connection at the periphery of the membrane. This design provides an improved fluid flow in the dressing by ensuring that fluid supplied is distributed over the entire surface of the first material body and hence over the entire wound bed, before it is aspirated over the periphery of the membrane. The membrane, at least on the side facing the first material body, preferably has a number of protuberances in order to create distribution channels, which facilitate the aforementioned distribution. A second body is preferably composed of a soft, liquid-permeable material arranged between the membrane and the covering layer, in order to facilitate the drainage of fluid, which is aspirated over the periphery of the membrane.

Said skin-friendly adhesive is applied to the covering layer at least in the area thereof that extends outside the contour of the first material body, on the side of the covering layer which, when applied, faces the skin.

In the first embodiment said skin-friendly adhesive is applied to one side of a film of plastic material, which on its opposite side is affixed to the covering layer in the area thereof that extends outside the contour of the first material body, on the side of the covering layer which, when applied, faces the skin.

In a first alternative the film coated with said skin-friendly adhesive comprises a central opening.

In a second alternative the film coated with said skin-friendly adhesive extends inside the area of the first material body and is perforated in this area.

The first and second material bodies are suitably composed of polymer foam.

Each connection for the drainage of fluid is connected to a vacuum pump, which may be of disposable type, for example a manual bellows pump, and comprises a vacuum chamber, which serves as storage chamber for the aspirated fluid.

In an especially advantageous embodiment the vacuum pump is pre-evacuated, so that a certain negative pressure prevails in the vacuum chamber and the connection for the drainage of fluid is closed. In such an embodiment the dressing is supplied as a unit with all constituent components connected to one another and the irrigation process will be performed automatically once the dressing has been applied and the connection for the drainage of fluid has been opened. The connections for the supply and drainage of fluid suitably consist of tubes, which extend between the covering layer and the first material body.

The invention also relates to a method of applying an irrigation dressing as described above to a wound, characterized by the following steps:

a) a central hole corresponding to the contour of the wound bed is cut out of a plastic film coated with a skin-friendly, sealing adhesive and the plastic film is affixed to the skin around the wound bed,
b) a unit including a first body of soft liquid-permeable material, an airtight and liquid-tight membrane and tubes for the supply and drainage of fluid, of which the tube or tubes for supplying fluid open out on one side of the membrane and the tube or tubes for drainage on the other side of the membrane, is applied to the wound bed with the first material body closest to the wound bed, so that said hole is entirely covered by the first material body,
c) a covering layer of flexible material is applied on top of said unit and is tightly affixed to the plastic film,
d) the tubes for the supply and drainage of fluid are connected to a reservoir for irrigation fluid and to a vacuum pump respectively, before or following the measures a-c.

The invention also relates to a further method of applying an irrigation dressing according to claim 1 to a wound, which irrigation dressing includes a first unit including a first body of soft liquid-permeable material, an airtight and liquid-tight membrane and tubes for the supply and drainage of fluid, of which the tube or tubes for supplying fluid open out on one side of the membrane and the tube or tubes for the drainage on the other side of the membrane, and a second unit consisting of an airtight and liquid-tight covering layer of flexible material, which on its underside has a coating of soft, skin-friendly adhesive, which affords sealing against micro-leakage, characterized by the following steps:

e) the first unit is trimmed so that the first body and the membrane assume a contour corresponding to the contour of the wound bed,
f) the first unit is applied to the wound bed with the first material body closest to the wound bed,
g) the covering layer coated with the adhesive layer is applied on top of said unit and is tightly affixed to the skin around the wound bed,
h) the tubes for the supply and drainage of fluid are connected to a reservoir for irrigation fluid and to a vacuum pump respectively, before or following the measures a-c.

### BRIEF DESCRIPTION OF DRAWINGS

The invention will now be described with reference to drawings attached, of which:

FIG. 1 schematically represents a cross section of a first embodiment of an irrigation dressing according to the invention,

FIG. 2 schematically illustrates the measurement of the adhesive strength against the skin,

FIG. 3 shows a cone for measuring softness,

FIG. 4 illustrates a method of measuring softness,

FIGS. 5-11 illustrate the MHC leakage test,

FIG. 12 shows the result of the MHC leakage test,

FIG. 13 shows the adhesive strength as a function of the time for a number of dressings,

FIG. 14 schematically represents the irrigation dressing according to FIG. 1 applied to a patient, and

FIG. 15 schematically illustrates a method of applying an irrigation dressing according to a second embodiment of the invention to a patient.

### MODE(S) FOR CARRYING OUT THE INVENTION

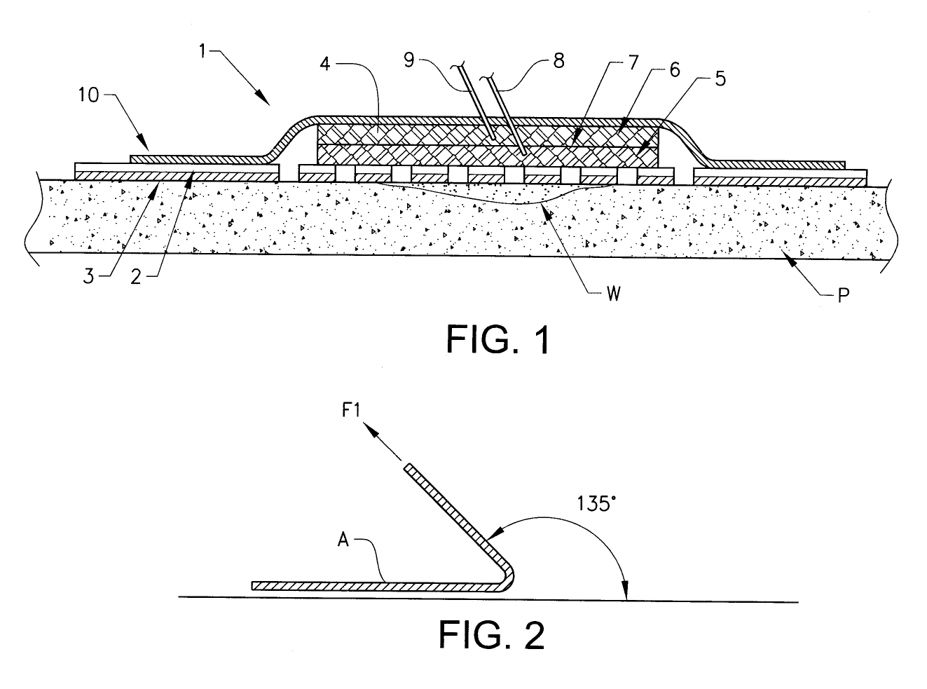

FIG. 1 schematically represents an irrigation dressing according to a first preferred embodiment of the invention applied over a wound W on a patient P. The first component of the dressing consists of a film 2 coated with a coating 3 of an adhesive, which affords sealing against micro-leakage.

As can be seen from FIG. 1, the film and its adhesive coating 3 are perforated within a central area, which extends over and around the wound W.

A primary function of the adhesive coating 3 is to tightly connect the dressing 1 to the patient's skin, so that the infiltration of air between the skin and the adhesive coating is prevented, and to securely affix the dressing to the skin so that the product remains in place under all the normal stresses to which the dressing is exposed.

The adhesive in the coating must also be skin-friendly and permit removal of the dressing without damaging the skin.

The adhesive may advantageously consist of a silicone elastomer, which is very soft and has a low surface energy, so that it moistens very effectively against the skin, that is to say it flows out into any irregularities in the skin and creates a large contact area between the skin and the silicone elastomer. This large contact area helps the silicone elastomer to attach well to the skin despite the fact that the bonding force of the silicone elastomer to the skin is inherently not that great. The adhesive strength represents a measure of the energy that is required in order to separate/pull the adhesive layer away from the skin. A contributory factor to the large amount of energy and hence the high stripping force that is required in order to remove the silicone elastomer from the skin, despite the relatively weak bonding strength, is the large amount of energy needed to stretch the soft silicone elastomer before it will detach from the skin. The softer and thicker the layer of silicone elastomer, the more force/energy that is needed to remove the elastomer from the skin.

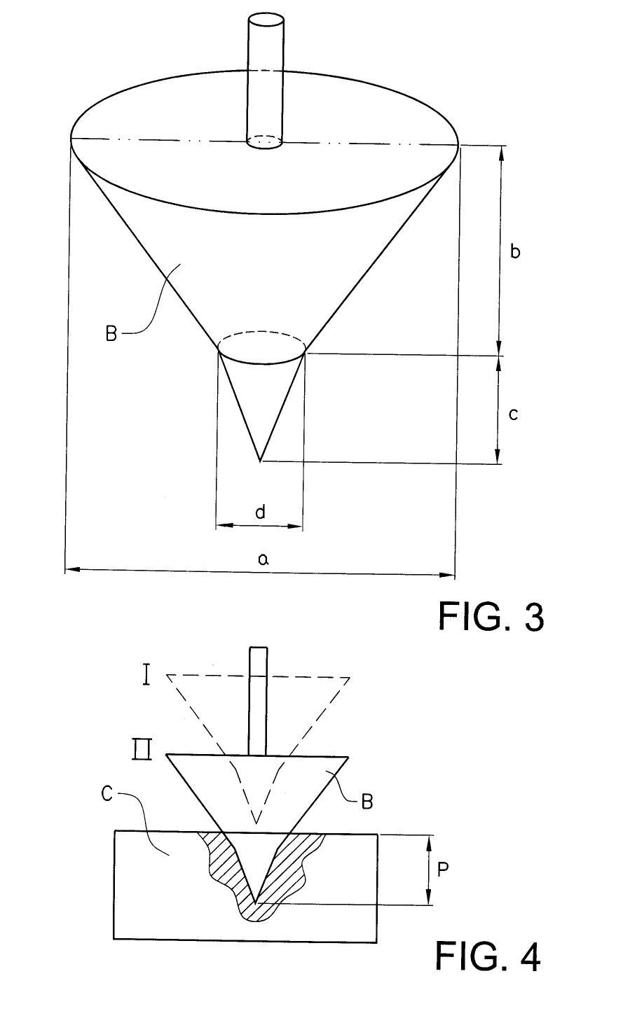

Since the skin characteristics vary from person to person, the capacity of the adhesive coating to adhere to the skin naturally also varies for different patients. The adhesive strength also varies as a function of the thickness of the soft adhesive and the mechanical characteristics of the carrier layer. The now standard methods for measuring adhesion use plates of different types, for example steel or glass, and do not provide values relevant for the measurement of skin adhesion. The values for the adhesive strength of an adhesive in contact with skin, which are specified below must be measured by means of a method which is illustrated schematically in FIG. 2 and which has been developed by the applicant.

Strips of a self-adhesive film dressing, the skin-adhesive strength of which is to be measured, are punched out to a size of 25×125 mm. It should be noted that all strips are also provided with a carrier layer on the rear side of the film dressing. (The function of this carrier layer is to stiffen the strips when applying them against the skin.) The strips are then applied to the skin on the backs of healthy human volunteers. The strips are carefully rubbed down with a finger and the backing carrier layer on the strips is then removed. Finally, the strips are pressed firmly against the skin for 3 seconds using a foam plastic sponge (42×182 mm, thickness=48 mm) firmly glued to a steel plate (50×200 mm, thickness=1 mm). The pressure is estimated a 6 kN/m². The strips are left on the skin for 2 minutes. The strips are then pulled off at a speed of 25 mm/sec and the stripping force F1 is measured. The stripping angle, that is to say the obtuse angle that is formed between the skin surface and the part of the strip removed must be 135°. The adhesive strength of the strip against the skin is represented by the mean force of the force F1.

Adhesives that can be used for the film dressing according to the invention must have an adhesive strength according to this method of at least 0.2-4 N/25 mm. The adhesive strength is preferably 1-2.5 N/25 mm.

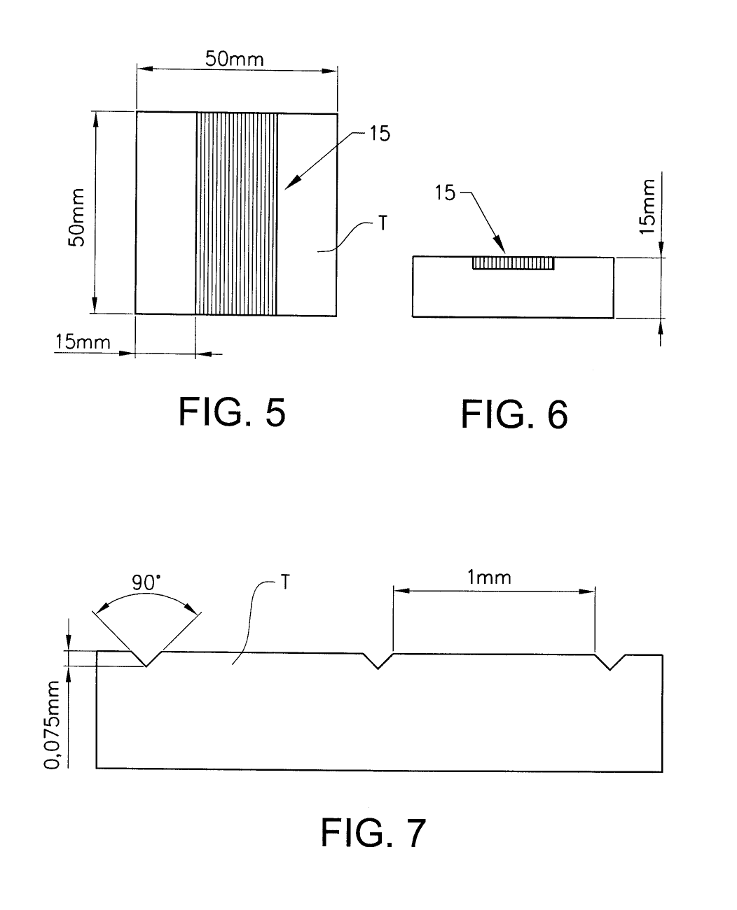

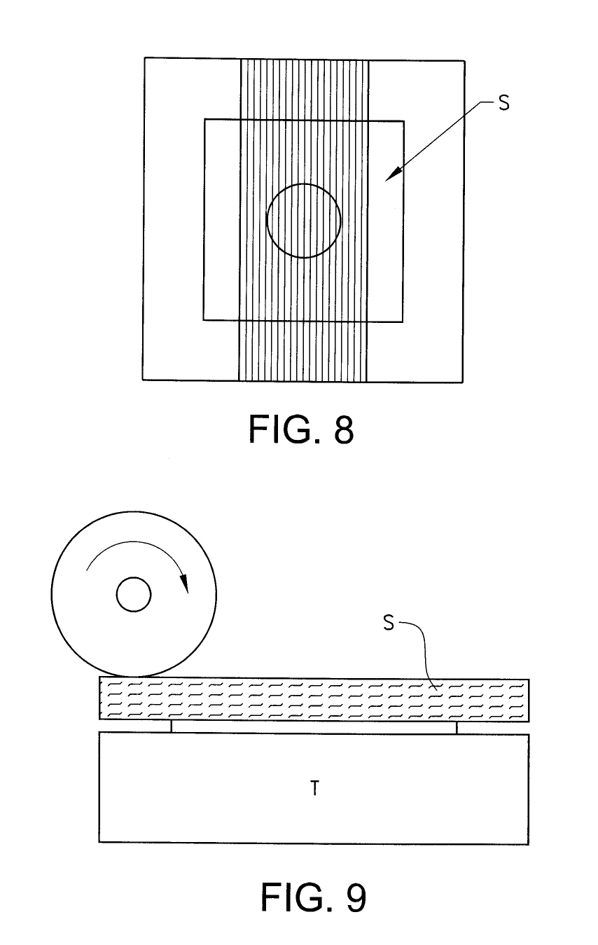

Adhesives according to the present invention must have a softness of more than 10 mm, measured by a method based on ASTM D 937 and ASTM D 51580. Certain changes, which are set out below, have been made. FIGS. 3 and 4 illustrate this modified method of measuring the softness of an adhesive by allowing a cone B with a weight of 62.5 g to press down under gravity into a 30 mm thick test body C of the adhesive, the softness of which is to be determined. The test body is produced by filling a cylindrical glass vessel with an inside diameter of 60 mm and an internal height of 35-40 mm with adhesive to a height of 30 mm. For a silicone elastomer, uncured silicone prepolymer is poured into the vessel before then being cross-linked to form an elastomer in the glass cylinder. The cone used is shown in FIG. 3 and has the following dimensions: a=65 mm, b=30 mm, c=15 mm and d=8.5 mm. The softness measuring method is conducted by first lowering the cone B down into a position I, which is shown by dashed lines in FIG. 4 and in which the tip of the cone just touches the surface of the test body C. The cone B is then released so that under the force of gravity it presses down into the test body C. The number of millimetres by which the tip of the cone has pressed into the test body after 5 seconds is measured and constitutes the penetration value P, which increases with the softness of the test body. The penetration value P constitutes the measure of softness that is used in the present invention. A PNR 10 penetrometer from Sommer & Runge K G, Germany, is used for performing the method.

It has emerged that even with soft, skin-friendly adhesive, which forms barriers preventing fluid flow through it, liquid and air can leak through these barriers via skin fissures, skin wrinkles or other irregularities in the skin. In analysing the leakproofing provided by film dressings, the applicant discovered an unexpected weakness in the conventional film dressings. In studies under the microscope it emerged that liquids can easily be diffused under the film dressings, despite the dressings seemingly being affixed by a thoroughly tight seal to the skin. Liquid proved to be capable of spreading several centimetres under the dressing via the naturally occurring microscopic folds on normal skin. Since the leakage consists of very small quantities and is not apparent from a study of the ingress of colourless liquids, this has previously been overlooked. The phenomenon, referred to as micro-leakage, could be observed only when the liquid was dyed with a highly coloured pigment.

It has surprisingly emerged that for a skin-friendly adhesive the aforementioned risk of leakage can be eliminated or at least significantly reduced if the adhesive is sufficiently soft and has a sufficiently high weight per unit area. It has also emerged that such adhesive also prevents the leakage of air through the adhesive barrier between the dressing and the skin.

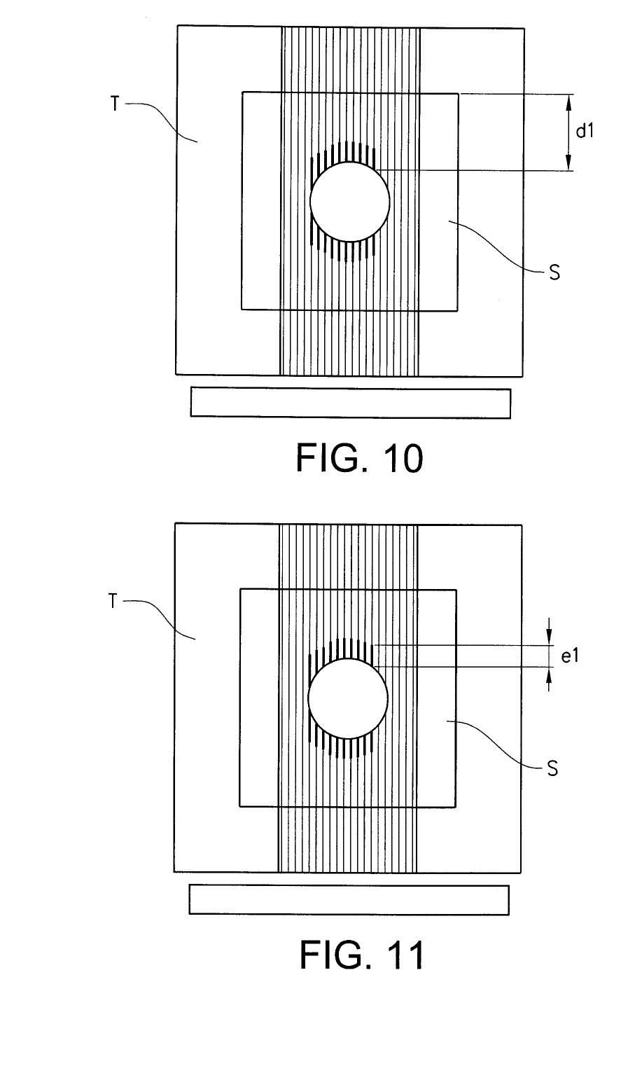

The method described below, known as the MHC leakage test, has been developed by the applicant in order to determine whether or not a film dressing is leakproof. Test specimens S 30×30 mm in size with a circular hole (diameter=12 mm) in the centre of the specimens are punched out of the dressing to be tested. Coloured test liquid is prepared by mixing 0.2% by weight of Patentblott V (from VWR International, Sweden) and 0.1% by weight of Teepol Gold (from Teepol Products, UK) with de-ionized water. An aluminium test plate T measuring 15×50×50 mm provided with 15 milled grooves is made up, see FIG. 5, which shows a plan view of the upper side of the plate, and FIG. 6, which shows a side view of the plate. For a more detailed description of the form of the grooves, see FIG. 7, which shows a cross sectional view of a part of the plate.

In FIG. 7 the groove depth is 75 micrometers, but other groove depths can also be used if it is intended to test the leakproofing against skin fissures or skin folds of some other depth, for example 50 micrometers or 150 micrometers.

A test specimen S is then carefully positioned centrally over the grooves in the test plate T in such a way that no air bubbles are produced between the test plate and the test specimen, see FIG. 8. No pressure must be exerted on the specimen when it is placed against the plate, so that if air bubbles occur these must not be forced away with the fingers, it being necessary instead to raise and reposition the specimen, or alternatively to discard it.

A piece of polyurethane foam (L00562-6, 1.6 mm from Rynel Inc., Boothbay, Me., USA) measuring 50×50 mm is then placed on top of the specimen S and the test plate T. A mangle made of metal (44 mm wide, r=48 mm, weight=995 g) is then rolled over the foam and the test specimen at a speed of 5 mm/second, see FIG. 9. The mangle is rolled back and forth once over the specimen.

The piece of foam is removed from the specimen 5 and 65 μl of test fluid is placed in the hole on the test specimen using a pipette. The test fluid is dispersed uniformly in the hole using the tip of the pipette, so that the fluid reaches every point on the edge of the specimen. A stop watch is started as soon as all the test fluid is evenly distributed in the hole. After 30 minutes a picture of the test specimen S is taken with a digital camera and the test fluid is placed on the test plate T together with a calibrated ruler.

The photograph taken is used to measure the following distances. For all the grooves in contact with the hole on the specimen, that is to say in all the grooves into which fluid may be expected to penetrate, the distance d from the edge next to the hole to the edge on the end of the specimen is measured, see FIG. 10, which shows this distance d1 for one of the grooves. All these distances d are then added together, and together they represent the total distance for which it is possible for the specimen to leak. After this, the distance e for which the test fluid has run in all the grooves on the plate is measured, see FIG. 11, which shows the distance e1 for one of the grooves. The combined length of all the distances e represents the total leakage distance.

Finally, the leakage is obtained by dividing the combined leakage distance e by the total distance d for which it is possible for the specimen to leak. This quotient is then converted to a percentage by multiplying by 100. The leakproofing is assessed as follows: Result>10% leakage, is counted as leakage, result≦10% is counted as leakproof.

Note that between each measurement on the test plate, the plate must be cleaned as follows. The plate is first rinsed with water, and is then washed with n-heptane. It is important to ensure that no adhesive residues remain in the grooves on the plate, and a soft material of the non-woven compress type (Mesoft® Mölnlycke Health Care) may be dipped in n-heptane and used to rub away adhesive residues in the grooves on the plate. Finally the plate must be left to dry in air before it can be reused.

Other solvents may be used for adhesives that are not soluble in n-heptane.

The reason why the test specimen should be analysed some time after application is that any leakage occurs with the aid of capillary forces, which means that it can be difficult, directly after application, to decide whether the test specimen is leakproof or not.

The test method described above with an aluminium plate groove depth of 75 micrometers has shown that a test body consisting of a transparent polyurethane film 25±5 micrometers thick, having an adhesive coating of a skin-friendly adhesive with a weight per unit area of approximately 50 g/m² and a softness of approximately 20 mm is leakproof according to this test. It has also emerged that a test specimen with such an adhesive coating is leakproof on normal, smooth skin of younger and middle-aged subjects. For areas of wrinkled skin, therefore, weights per unit area in excess of 50 g/m² may be necessary in order to ensure leakproofing.

The way in which leakproofing is affected by the softness and weight per unit area of the adhesive in the adhesive coating has been analysed by the aforementioned method for a silicone elastomer, Silgel 612 from Wacker Chemie GmbH, Germany.

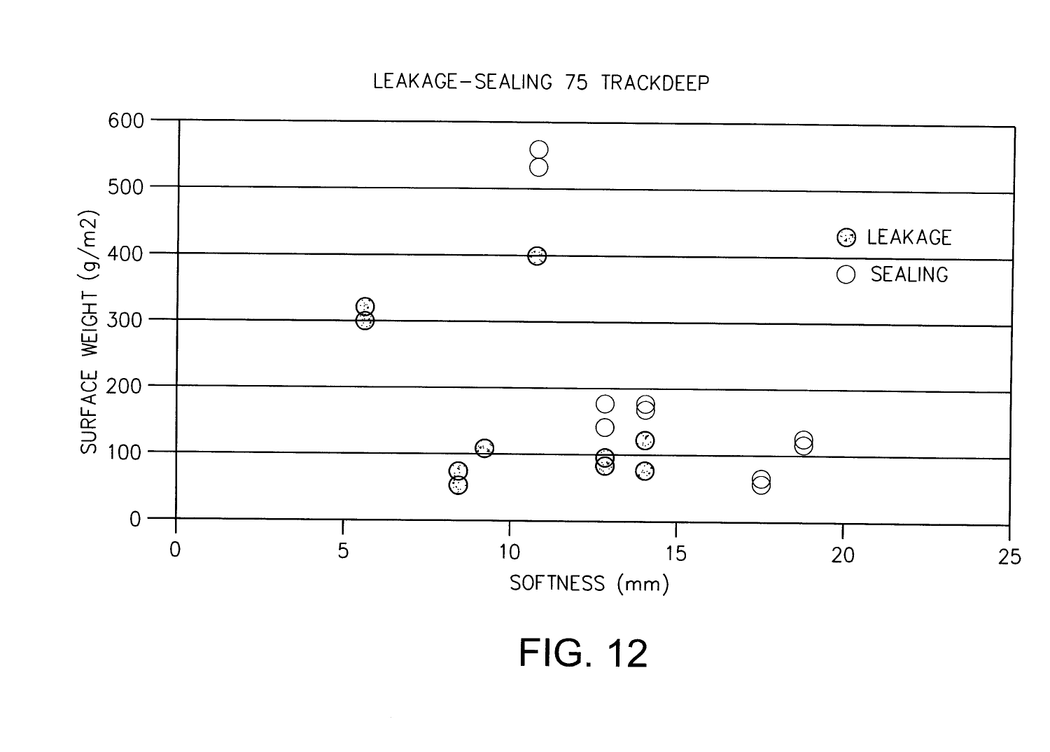

In an MHC leakage test with a groove depth of 75 micrometers the leakage has been measured for a number of different film dressings with adhesive of different softnesses and weights per unit area. All dressings were manufactured by coating a 25±5 micrometer thick polyurethane film with Silgel 612 of different softnesses and weights per unit area. The result is shown in FIG. 12.

The results clearly show that there is a correlation between softness (penetration) and weight per unit area of the silicone elastomer. The softer the silicone elastomer, the lower the weight per unit area required for sealing. The results indicate that, given a sufficient number of measurements, it is possible to produce a curve that describes precisely the minimum weight per unit area that is required at a given softness to ensure sealing against the skin. The results make it clear that such a curve has a steep initial incline, that is to say in the case of less soft adhesives, after which it levels out. The correlation between weight per unit area and softness is therefore such that very soft adhesives are required in order to achieve tight sealing at low weights per unit area, whereas less soft adhesives require higher weights per unit area in order to achieve tight sealing. It is obvious that, at a softness of less than 10 mm, it is difficult, and perhaps even impossible, to achieve liquid-tight film dressings. At softness values in the order of 20 mm a weight per unit area of 50 g/m² may be sufficient to achieve sealing. It has emerged that the soft adhesives, which afford sealing against micro-leakage, also afford sealing against air leakage.

It should be added that all known film dressings that were tested leaked in respect both of fluid and of air.

As can be seen from FIG. 12, certain points merge, since several of the film dressings tested had approximately the same weights per unit area and softness.

Besides increasing the leakproofing, a greater weight per unit area of the adhesive coating affords a reduced risk of blisters, spots or other lesions occurring on the skin at the edges of the applied adhesive. Such lesions can occur under the movements of the film dressing carrier, which lead to relative movement between the skin and the adhesive coating or due to the fact that the dressing is stressed by external forces, for example if the dressing carrier rests against an object. It has emerged that the risk of such lesions occurring diminishes with a higher weight per unit area and greater softness of the adhesive coating. This is probably due to the fact that a proportion of the stress loading is absorbed by the adhesive layer through deformation and is thereby not transmitted to the skin. The dressing according to the invention can furthermore stretch together with the skin, which reduces the risk of a shearing action occurring between the skin and the adhesive, which can cause mechanical damage to the skin.

In order to ensure that only a light application force is needed when applying dressings according to the present invention, the softness of the soft, skin-friendly adhesive used is preferably greater than 10 mm, preferably between 12 and 17 mm. The softer the adhesive, the more rapidly it flows into any irregularities in the underlying surface, which means that the dressing according to the present invention is more leakproof against both fluid and air immediately after application on normal skin. With a softness of more than 17 mm there is a risk that the internal cohesion of the adhesive will be insufficient, so that adhesive residues are left on the skin when an applied dressing is removed.

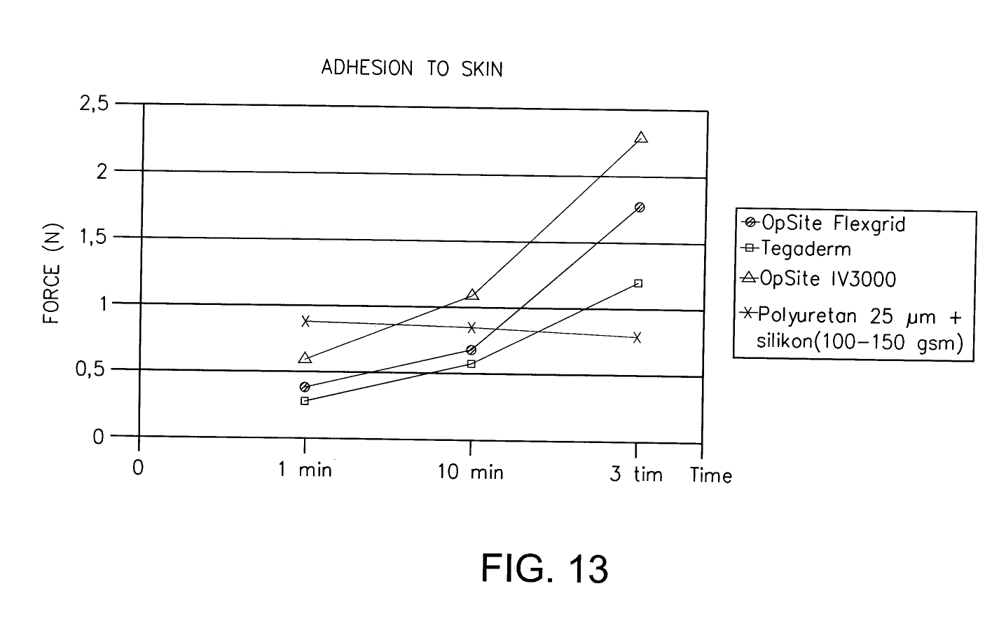

Another important characteristic is that the adhesive strength of the soft, skin-friendly adhesive used in dressings according to the invention does not vary over time or only varies to a slight degree over time. This has been verified by measuring the adhesive strength against the skin for a number of known film dressings and a film dressing with an adhesive according to the invention containing silicone elastomer. The known dressings were Tegaderm™ from 3M Health Care, USA; OpSite™ IV3000™ and OpSite™ Flexigrid™ from Smith & Nephew Medical Limited, England. Measurement was performed by the method of measuring skin adhesive strength described above, with the difference that the measurements were performed after 1 minute, 10 minutes and 3 hours. The results are shown in FIG. 13. As can be seen from the figure, the adhesive strength increased sharply over time for the known film dressings, whilst the dressing according to the invention basically exhibited a constant adhesion. In figures, the adhesive strength increased by 295% from 1 minute to 3 hours for OpSite™ Flexigrid™, 209% for Tegadem™ and 318% for OpSite™IV3000™.

The adhesive layer 3 is advantageously made up of a silicone composition, which after mixing together crosslinks to form a soft elastomer. RTV (Room Temperature Vulcanizing) silicone systems, which are addition-curing and which can be cross-linked at moderate temperatures, are especially suitable. RTV silicones can be made soft, sensitive and self-adhesive.

Examples of RTV addition-curing silicon systems are given in EP 0 300 620 A1, which describes so-called “gel-forming compositions”, composed of an alkenyl-substituted polydiorganosiloxane, an organosiloxane containing hydrogen atoms bonded to a proportion of the silicone atoms and a platinum catalyst.

An example of a commercially available RTV silicone system is Wacker SilGel 612 from Wacker-Chemie GmbH, Munich, Germany. This is a two-component system. By varying the proportions between the two components A:B from 1.0:0.7 to 1.0:1.3 it is possible to vary the softness and the level of adhesion of the elastomer formed.

Examples of other soft silicone elastomers that are adhesive on dry skin are NuSil MED-6340, NuSil MED3-6300, NuSil MED12-6300 from NuSil Technology, Carpinteria, Ga., USA and Dow Corning 7-9800 from Dow Corning Corporation, Midland, USA.

It is also feasible to use soft, hot melt adhesives. One example of such an adhesive is Dispomelt 70-4647 from National Starch, USA.

In the embodiment shown in FIG. 1 a component 4 comprising two bodies 5, 6 of a soft fluid-distributing material, preferably a foam material, such as polyurethane foam, a membrane 7 of an airtight and liquid-tight material arranged between these bodies 5, 6 and two tubes 8, 9 for the supply and drainage of fluid, are situated on top of the film 2. The supply tube 8 opens out beneath the membrane 7 and the drainage tube 9 opens out on top of the membrane. The terms “on top of”, “beneath” and similar positional terms are relative and refer to FIG. 1, in which the dressing is placed on the upper side of a patient. The relative positions of the components in the dressing would be the same, even if the dressing were placed on the underside of a patient's arm, for example.

Extending on top of the component 4 is a covering layer 10, preferably a plastic film, for example a polyurethane plastic. The covering layer 10 extends over the entire component 4 and also outside the contour thereof. The part of the covering layer 10, which extends outside the contour of the component 4 is tightly affixed to the upper side of the film 2, for example by adhesive bonding or hot welding.

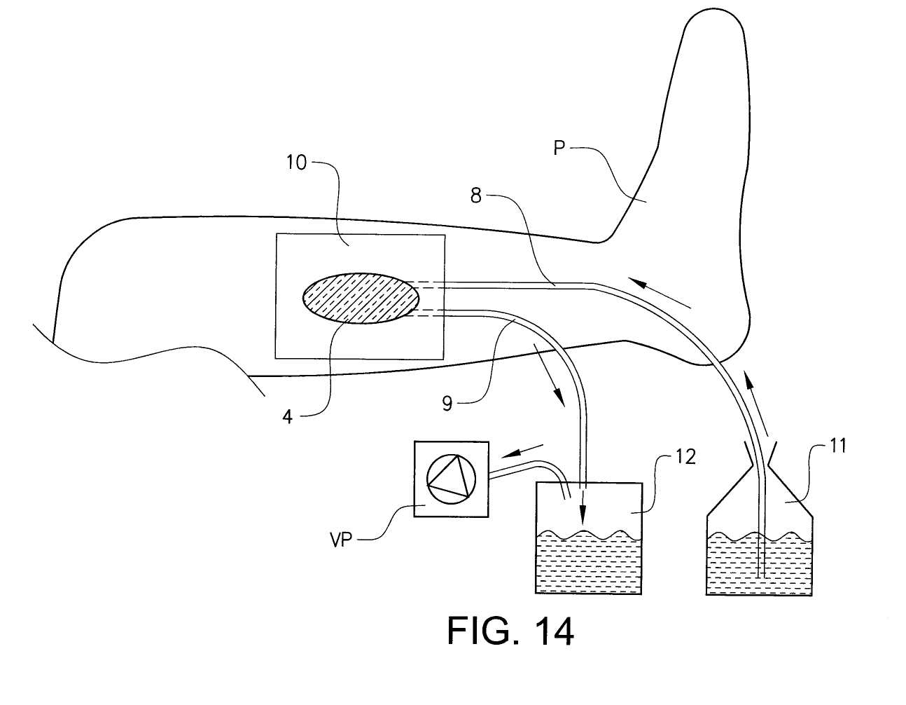

FIG. 14 shows a schematic representation of the dressing in FIG. 1, applied to the leg of a patient P. The tub 8 is connected to a reservoir 11 for irrigation fluid and the tube 9 to a vacuum pump VP via a collecting vessel 12.

The dressing functions as follows:

When a negative pressure is generated in the tube 9 by means of the pump VP, fluid present in the dressing will be aspirated into the tube 9, which means that a negative pressure is also generated in the tube 8. Irrigation fluid from the reservoir 8 will then flow into the dressing and will be distributed in the foam body 5, which of the two foam bodies 5, 6 lies closest to the wound, and will flow over the wound bed on its way to the periphery of the membrane 7. The irrigation fluid mixed with excess exudates from the wound bed will then flow round the periphery of the membrane 7 and will be aspirated into the other foam body 6, situated on the opposite side of the membrane to the first foam body 5 and then on into the tube 9.

The reservoir 8 with irrigation fluid is preferably closed to the surrounding atmosphere and at the outset is at atmospheric pressure. The reservoir 8 is furthermore preferably formed from a flexible material, so that it is compressed by the atmospheric pressure as the irrigation fluid is aspirated out of the reservoir. Another possibility is to have the reservoir open to the atmosphere and suitably provided with a filter, which prevents airborne bacteria or other pathogens being entrained into the reservoir with the air. In the preferred embodiment of the invention, the vacuum pump VP functions as a vacuum vessel, that is to say the vacuum pump is not continuously activated, but the negative pressure in the vessel 12 is instead reduced as it is gradually filled with fluid. The flow through the dressing will cease when the pressure in the irrigation fluid reservoir 8 has fallen to such a degree that the pressure differential between the negative pressure in the vessel 12 and the pressure (almost atmospheric pressure) in the reservoir 8 has been equalized. The negative pressure generated and the flow resistance in the system are designed so that the rate of flow is low, preferably 0.5-100 ml per hour. This means that the fluid supplied through the tube 8 seeps rather than flows into the space between the membrane 7 and the foam body 5 and that a correspondingly small quantity of fluid plus wound fluid exuded from the wound is aspirated around the periphery of the membrane 7 and into the space on top of the membrane, before then being drained through the tube 9. The low rate of flow means that the fluid supplied to the space below the membrane spends a relatively long time in the space, which means that it can easily be distributed into the foam body and reach the actual wound bed. The low rate of flow inside the dressing also ensures that the fluid delivered manages to penetrate down to the wound bed before it reaches the periphery of the membrane.

It is naturally possible, by reactivating the pump VP, to produce a new negative pressure when the pressure differential between the vessel 12 and the reservoir 11 becomes too small, but because the irrigation fluid passes through the dressing so slowly, the fluid flow can continue for a period of at least eight hours without the need to reactivate the pump. This makes it possible to use vacuum pumps of disposable type, for example manual bellows pumps, which can be discarded together with the dressing after use. In an advantageous variant, in one such application the vacuum chamber of the bellows pump is used as storage vessel for the aspirated fluid, that is to say the tube 9 in FIG. 14 opens out directly into the vacuum chamber of a bellows pump. In such an application the negative pressure and the dressing are suitably designed so that the fluid flow continues throughout the active time of the dressing, that is to say throughout the entire period for which the dressing is to be applied. The irrigation process then takes place automatically after application of the dressing, without the need for any action on the part of staff or patients.

It is furthermore possible, instead of a pump, to connect the tube 9 to a vacuum vessel, in which a certain negative pressure prevails. The vacuum vessel may also consist of a pre-evacuated bellows pump, or a bellows pump with markings or stops, which denote the intended compression of the pump in order to obtain a specific negative pressure. If a vacuum vessel, pre-evacuated bellows pump or the like is used, the tube 9 is suitably connected to the vacuum chamber, i.e. the space subjected to a negative pressure, by pricking a hole in a membrane or the like, which seals off the vacuum chamber, prior to use, for example by inserting the tube 9 or by twisting or other movement of the tube, if the dressing is constructed as an integral unit together with the irrigation fluid reservoir and the vacuum source.

In a variant the supply tube is provided with a valve, which allows the tube to be closed, so that the irrigation fluid reservoir can be changed without losing the negative pressure in the wound bed. An empty irrigation reservoir can thereby be replaced with a new one, or a reservoir with one type of irrigation fluid can also be replaced with another reservoir holding another type of irrigation fluid. It is naturally also possible to vary the negative pressure by compressing the bellows pump if the pressure has fallen too much, or if a greater negative pressure is required for some other reason. If a vacuum vessel is used on the dressing instead of a bellows pump, this can suitably be provided with a closing valve, to allow the dressing to be changed without wasting the negative pressure remaining in the vessel.

It is pointed out that for a dressing according to the preferred embodiment described above to function in the preferred manner described above, the adhesive coating 3 must ensure sealing against micro-leakage of air throughout the effective life of the dressing. It is also pointed out that the dressing must naturally also be tight in respect of macro-leakage. If the skin should have exceptionally deep fissures, it may therefore be necessary to seal such fissures before the dressing is applied. This is suitably done by means of a silicone composition, which is highly viscous when applied to the skin and thereby fills such fissures and which then cures at room temperature to a soft, skin-friendly silicone elastomer. Such a silicone composition is disclosed by WO 2004/108175 A1.

The irrigation fluid may advantageously contain substances which promote healing or are otherwise beneficial, such as antibiotics, antimicrobial substances, antiseptics, growth factors, pH buffers (e.g. acidifying), salts (such as physical NaCl solution, for example), antioxidants, vitamins, enzymes (e.g. proteolytic enzymes), and nutrients.

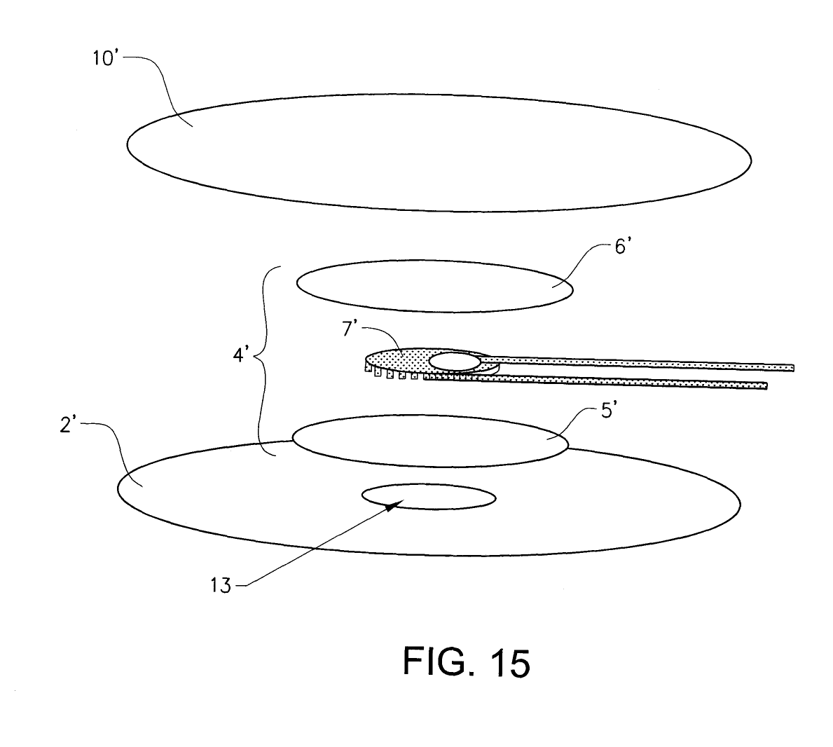

FIG. 15 shows a second embodiment of the invention, which differs from the embodiment shown in FIGS. 1 and 14 primarily in that the dressing in FIG. 15 has a somewhat differently formed membrane 7′. The components of the dressing that correspond to similar components in the embodiment shown in FIG. 1 have been given the same reference numerals with the addition of a prime sign.

Like the film 2 in the embodiment shown in FIG. 1, the film 2′ is provided with a coating of a soft, skin-friendly adhesive, which affords sealing against micro-leakage (not shown in FIG. 14). In its central part the film 2′ has an aperture 13, which extends at least round the wound bed and leaves this open. The component 4, comprising bodies 5′, 6′ of liquid-permeable material, the liquid-tight membrane 7′ and the tubes 8′, 9′ differ from corresponding components in FIG. 1 primarily in that the membrane 7′ is provided with a pattern of protuberances, which extend on the underside of the membrane 7′. The protuberances in FIG. 14 are cylindrical in shape but may take any shape including a linear shape. The object of the protuberances is to ensure that fluid can be distributed over the entire surface of the underlying material body, even if this should become locally clogged, for example by particles or the like from the wound bed. In order to achieve this, it is not always necessary to form protuberances, and the choice of a material having an uneven surface texture, such as a textile or non-woven material, for the membrane 7′, may instead be sufficient.

The ends of the tubes 8′, 9′ are furthermore integrally joined to the membrane, so that the membrane and the tubes can be applied as one unit. The material bodies 5′, 6′ may also be joined to the membrane.

Like the dressing shown in FIG. 1, the dressing shown in FIG. 15 may be supplied with all components joined together into one unit, although it can also be supplied with the components 2′, 4′ and 10′ designed as separate units. The adhesive layers on the covering layer 10′ and the film 2′ are then covered by a release layer, such as polyethylene film, and a stiffening layer is detachably fastened to the upper sides of the films. Such an embodiment permits precise adaptation of the dressing to the shape of the wound to which it is to be applied. Such a dressing is applied as follows:

a) a central hole corresponding to the contour of the wound bed is first cut out of the plastic film 2′ coated with a skin-friendly, sealing adhesive and the plastic film 2′ is affixed to the skin around the wound bed (the release layer is preferably removed after cutting to size), following which
b) the unit 4′ including a first body 5′ of soft liquid-permeable material, an airtight and liquid-tight membrane 7′ and tubes 8′, 9′ for the supply and drainage of fluid, of which the tube 8′ for supplying fluid opens out on one side of the membrane and the tube 9′ or tubes for the drainage on the other side of the membrane, are applied to the wound bed with the first material body 5′ closest to the wound bed, so that said hole is entirely covered by the first material body, following which
c) the covering layer 10′ of flexible material is applied on top of said unit and is tightly affixed to the plastic film, following which
d) the tubes for the supply and drainage of fluid are connected to a reservoir for irrigation fluid and to a vacuum pump respectively, before or following the measures a-c.

In an embodiment of the dressing (not shown) this is without the lower film (the film 2, 2′ in the embodiments shown) and the soft, skin-friendly adhesive is directly affixed to the underside of the covering layer. In this embodiment, too, the covering layer constitutes a first unit, which is separate from a second unit comprising foam bodies, membrane and tubes. The second unit is preferably also a physical unit, that is to say the constituent components are joined to one another. Such a dressing . . . as follows:

a) the second unit comprising foam bodies, membrane and tubes is trimmed so that it assumes a contour corresponding to the contour of the wound bed (naturally without cutting of the tubes), following which
b) the second unit is applied to the wound bed with the first foam body closest to the wound bed, following which
c) the covering layer coated with the adhesive layer is applied on top of the second unit and is tightly affixed to the skin around the wound bed, following which
d) the tubes for the supply and drainage of fluid are connected to a reservoir for irrigation fluid and to a vacuum pump respectively, before or following the measures a-c.

If the skin around the wound presents “macro fissures”, these are suitably filled with the aforementioned highly viscous silicone composition, which then cures in situ. The dressing must then be applied before such a composition has fully cured.

In the embodiment shown in FIG. 1 a perforated layer of adhesive 3 is applied closest to the wound bed. Such a layer is not adhesive against the wound bed. In the embodiments in which the adhesive layer 3 is only applied outside the wound bed, it is appropriate to coat the foam body 5, 5′ with a layer which is not adhesive to the wound bed. Such a layer must naturally be liquid-permeable and may consist of a reticulate layer, a perforated layer or a layer which, for example, has been sprayed over the entire surface of the foam body. The adhesives which are suitable for the adhesive layer 3 may also be used to produce layers that are not adhesive to the wound bed. Naturally, however, they need not have the softness or the weight per unit area that is necessary in the layer 3, which is intended to be resistant to micro-leakage.

As indicated in FIGS. 14 and 15, the membrane 7, 7′ and the tubes 8, 8, 8′, 9′ suitably form an integral unit, which may be integrally moulded, for example, or otherwise joined together to form a physical unit.

The covering layer 10, 10′ is composed of a thin, highly flexible material, such as polyurethane film with a thickness of less than 50 micrometers. Other plastic materials may also be used.

The film 2, 2′ is also composed of a thin, soft and sensitive material, such as polyurethane with a thickness of less than 50 micrometers. In the embodiments shown the material body 5, 5′ is composed of a soft, open-cell polyurethane foam. It is also feasible, however, to use other liquid-permeable materials, which are so deformable that they can conform to the shape of the wound bed, so that they will rest against this or against any underlying perforated film layer. Examples of such material are cotton wool, non-woven and textile material. The material used is preferably hydrophilic material.

The membrane may consist of a thin sheet of rubber or plastic material, such as polyurethane film with a thickness of 25-50 micrometers. It is also feasible to use a liquid-tight and airtight textile material or laminate of one or more textile materials or one or more plastic materials. It should be pointed out that the thickness relates to the wall thickness and not to the thickness of the membrane inclusive of any protuberance. Including the height of the protuberances shown in FIG. 15, the membrane may have a thickness of 1-3 mm.

The material body 6, 6′ may be composed of any of the materials that are suitable for the body 5, 5. The prime function of the body 6, 6′ is to prevent the opening of the tube 9 or the inlet to this on the top of the membrane 7, 7′ being closed by the covering layer 10, 10′ pressing against the membrane due to the pressure differential between the atmosphere outside the dressing and the negative pressure inside the dressing. It is therefore feasible, for example, to replace the material body 6, 6′ with a sheet of flexible material, which on its side facing the membrane has a peripheral annular channel and a number of channels which lead to the orifice of the tube 9.

The tubes are composed of polyurethane, silicone or other tube material commonly used for medical items. The inside diameter of the drainage tube 9, 9′ is 0.15-2 mm and of the supply tube 8, 8′ 1-10 mm. It must be noted that the drainage tube preferably has a larger inside diameter than the supply tube.

In the embodiments according to FIGS. 14 and 15, the tubes run out of the dressing between the covering layer 10, 10′ and under the film 2, 2′. It is naturally possible, instead, to have the tubes running through the covering layer. In such an application of a dressing, which is to be applied by the methods described above, that is to say a dressing in which the covering layer is a separate unit which is fitted in the final stage of the application process, the covering layer is suitably divided into two or more parts and is designed so that it can form a seal around the tubes when applied.

It is pointed out that even though the tubes in the drawings are shown extending from the membrane to the pump and the irrigation fluid reservoir respectively, the tubes may comprise multiple connectable parts. A tube connection part may furthermore be connected to the membrane, for example integrally formed with the membrane, the tubes being coupled together with the connection part at the time of application.

A dressing according to the embodiment in FIG. 1 has been tested in conjunction with a Bellovac pump (bellows pump). The dressing was applied to a test plate of the same type as in the MHC leakage test, having a groove depth of 75 micrometers. The bellows pump was then compressed so that a negative pressure of 18.67 kPa (140 mmHg) was produced in the drainage tube, following which irrigation fluid was made to flow through the dressing. The mean flow rate of the irrigation fluid was approximately 4.5 ml/hour. After 24 hours the negative pressure had been reduced by 25-40%. It was apparent from the test that the dressing was tight, so that no air infiltrated, the pressure reduction having been due solely to the transfer of fluid from the irrigation reservoir.

A dressing according to the embodiment in FIG. 1 has also been tested affixed to a person for 4 to 6 hours respectively. In this case, also, the pressure reduction was slight, approximately 10%.

The products referred to in the present invention are normally supplied sterile-packed, which means that the adhesives used must naturally be sterilizable in just the same way as other components in the actual dressing and the tubes.

The embodiments described can naturally be modified without departing from the scope of the invention. For example, the dressing may include more than one connection for the supply of irrigation fluid and/or more than one connection for the drainage of irrigation fluid. Pumps other than those described can also be used and it is also feasible to use control equipment, which controls the pump or pumps, so that a specific negative pressure always prevails in the connection or connections for the drainage of fluid. The adhesive affording a seal against micro-leakage may furthermore be applied directly to the underside of the covering layer. The tubes which lead to the pump and the irrigation fluid reservoir may be designed with multiple interconnecting parts and such a coupling in a tube part arranged closest to the vacuum source may be such that a sealing of this tube part is broken when coupling together, for example by perforating a membrane at the end of the tube. The irrigation fluid reservoir may be of the bag type or bottle type. If the irrigation fluid reservoir is of the bottle type, it is open to the atmosphere and preferably provided with a filter against airborne impurities. The dressing may contain a separate tube or the like for supplying wound treatment substances. Such a tube may extend to the underside of the membrane or may consist of a branch of the supply tube. The supply and drainage tubes, at least in proximity to the actual dressing, may be integrated into one single unit, that is to say a tube, which is divided by a dividing wall into two ducts. The scope of the invention will therefore be defined solely by the content of the patent claims attached.

1. Irrigation dressing, comprising a first body (5; 5′) of a soft, liquid-permeable material, at least one connection (8; 8′) for the supply of fluid, at least one connection (9; 9′) for the drainage of fluid, an airtight and liquid-tight covering layer (10; 10′) of flexible material, which covers said material body and extends outside the sides thereof, and means (2, 3; 2′) for affixing the area of the covering layer that extends outside the contour of the first material body to the skin, the first material body facing the wound bed (W) when the dressing is applied, characterized in that the means (2, 3; 2′) for affixing said area to the skin includes a layer (3) of a soft, skin-friendly adhesive, which affords sealing against micro-leakage and has a softness of more than 10 mm and a weight per unit area of at least 50 g/m².

2. Irrigation dressing according to claim 1, characterized in that the adhesive is leakproof according to the MHC leakage test with a groove depth of 75 micrometers.

3. Irrigation dressing according to claim 2, characterized in that the adhesive is applied to a plastic film (2; 2′) having a thickness of less than 50 micrometers.

4. Irrigation dressing according to claim 1, characterized in that said adhesive consists of an RTV addition-curing silicone system.

5. Irrigation dressing according to claim 4, characterized in that the membrane (7; 7′) of liquid-tight material is arranged between the covering layer (10; 10′) and the first material body (5; 5′), one or more connections (8; 8′) for the supply of fluid opening out between the membrane and the first material body (5; 5′), one or more connections (9; 9′) for the drainage of fluid opening out between the covering layer and the membrane, and that the space between the covering layer and the membrane is connected to the space between the membrane and the first material body via at least one connection at the periphery of the membrane.

6. Irrigation dressing according to claim 5, characterized in that the membrane (7′), at least on the side facing the first material body (5′), has a number of protuberances.

7. Irrigation dressing according to claim 6, characterized in that a second body (6; 6′) of a soft, liquid-permeable material is arranged between the membrane (7; 7′) and the covering layer (10; 10′).

8. Irrigation dressing according to claim 1, characterized in that said skin-friendly adhesive is applied to the covering layer at least in the area thereof that extends outside the contour of the first material body, on the side of the covering layer which, when applied, faces the skin.

9. Irrigation dressing according to claim 1, characterized in that said skin-friendly adhesive is applied to one side of a film of plastic material (2; 2′), which on its opposite side is affixed to the covering layer (10; 10′) in the area thereof that extends outside the contour of the first material body (5; 5′), on the side of the covering layer which, when applied, faces the skin.

10. Irrigation dressing according to claim 9, characterized in that the film (2′) coated with said skin-friendly adhesive comprises a central opening.

11. Irrigation dressing according to claim 9, characterized in that the film (2) coated with said skin-friendly adhesive extends inside the area of the first material body (5) and is perforated within this area.

12. Irrigation dressing according to claim 1, characterized in that said soft material bodies (5, 6; 5′, 6′) are composed of polymer foam.

13. Irrigation dressing according to claim 1, characterized in that each connection (9) for the drainage of fluid is connected to a vacuum pump (VP).

14. Irrigation dressing according to claim 13, characterized in that the vacuum pump (VP) is of disposable type and comprises a vacuum chamber, which serves as storage chamber for the aspirated fluid.

15. Irrigation dressing according to claim 14, characterized in that the vacuum pump (VP) is a manual bellows pump.

16. Irrigation dressing according to claim 14, characterized in that the vacuum pump (VP) is pre-evacuated, so that a certain negative pressure prevails in the vacuum chamber and the connection for the drainage of fluid is closed.

17. Irrigation dressing according to claim 1, characterized in that the connections for the supply and drainage of fluid consist of tubes (8′, 9′), which extend between the covering layer (10′) and the first material body (5′).

18. Irrigation dressing according to claim 1, characterized in that the connections for the supply and drainage of fluid consist of tubes (8, 9), which extend through the covering layer (10).

19. Method of applying an irrigation dressing according to claim 1 to a wound, characterized by the following steps:
h) a central hole corresponding to the contour of the wound bed is cut out of a plastic film coated with a skin-friendly, sealing adhesive and the plastic film is affixed to the skin around the wound bed)
i) a unit including a first body of soft liquid-permeable material, an airtight and liquid-tight membrane and tubes for the supply and drainage of fluid, of which the tube or tubes for supplying fluid open out on one side of the membrane and the tube or tubes for the drainage on the other side of the membrane, is applied to the wound bed with the first material body closest to the wound bed, so that said hole is entirely covered by the first material body,
j) a covering layer of flexible material is applied on top of said unit and is tightly affixed to the plastic film,
k) the tubes for the supply and drainage of fluid are connected to a reservoir for irrigation fluid and to a vacuum pump respectively, before or following the measures a-c.

20. Method of applying an irrigation dressing according to claim 1 to a wound, which irrigation dressing includes a first unit including a first body of soft liquid-permeable material, an airtight and liquid-tight membrane and tubes for the supply and drainage of fluid, of which the tube or tubes for supplying fluid open out on one side of the membrane and the tube or tubes for drainage on the other side of the membrane, and a second unit consisting of an airtight and liquid-tight covering layer of flexible material, which on its underside has a layer of soft, skin-friendly adhesive, which affords sealing against micro-leakage, characterized by the following steps:
l) the first unit is trimmed so that the first body and the membrane assume a contour corresponding to the contour of the wound bed,
m) the first unit is applied to the wound bed with the first material body closest to the wound bed,
n) the covering layer coated with the adhesive layer is applied on top of said unit and is tightly affixed to the skin around the wound bed,
d) the tubes for the supply and drainage of fluid are connected to a reservoir for irrigation fluid and to a vacuum pump respectively, before or following the measures a-c.
